(19) United States
(12) Patent Application Publication
(10) Pub. No.: US 2020/0010754 A1
(43) Pub. Date: Jan. 9, 2020

(54) METHOD FOR INHIBITING THE AGGLOMERATION OF GAS HYDRATES

(71) Applicant: ECO INHIBITORS AS, Stavanger (NO)

(72) Inventor: Malcolm Andrew Kelland, Stavanger (NO)

(21) Appl. No.: 16/491,019

(22) PCT Filed: Mar. 6, 2018
(86) PCT No.: PCT/GB2018/050563

§ 371 Date: Sep. 4, 2019

(30) Foreign Application Priority Data
Mar. 7, 2017 (GB) 1703615.3

(51) Int. Cl. C09K 8/52; C07C 237/06

(52) U.S. Cl. CPC C09K 8/52; C09K 2208/22; C07C 237/06

(57) ABSTRACT

A method for inhibiting the formation or agglomeration of gas hydrates in a system, said method comprising adding to the system a compound of formula (I) or a zwitterionic form thereof wherein R₁ is a group comprising 4 to 28 carbon atoms and at least one C═C double bond; L₁ and L₂ are independently selected from bonds and linker moieties containing 1 to 12 carbon atoms; and R₂, R₃, R₄, R₅, R₆, R₇, R₈, R₉ and R₁₀ are independently H or a group comprising 1-20 carbon atoms.

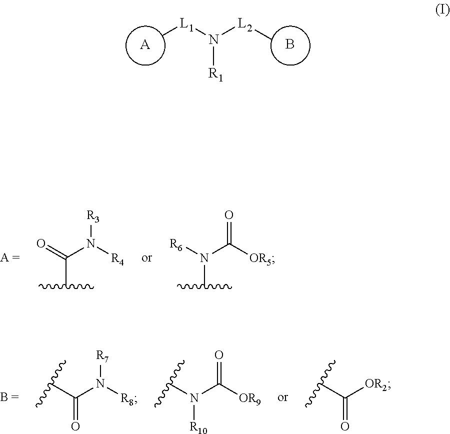

The present invention relates to biodegradable clathrate hydrate inhibitors and methods for inhibiting the nucleation, formation, agglomeration, and deposition of clathrate hydrates. The invention is especially useful in inhibiting blockages due to clathrate hydrates in pipelines for production and transport of oil and natural gas, in drilling operations, completion, stimulation and fracturing operations, and in injection and re-injection operations.

Gas hydrates are clathrates (inclusion compounds) of small molecules in a lattice of water molecules. In the petroleum industry, natural gas and petroleum fluids contain a variety of these small molecules, which can form gas hydrates. They include hydrocarbons such as methane, ethane, propane, isobutane as well as nitrogen, carbon dioxide and hydrogen sulphide. Larger hydrocarbons such as n-butane, neopentane, ethylene, cyclopentane, cyclohexane and benzene are also hydrate-forming components. When these hydrate-forming components are present with water at elevated pressures and reduced temperatures, the mixture tends to form gas hydrate crystals. For example, ethane at a pressure of 1 MPa forms hydrates only below 4° C., whereas at 3 MPa gas hydrates can form up to 14° C. These temperatures and pressures suited to hydrate formation are typical operating environments where petroleum fluids are produced and transported and in drilling, completion or fracturing operations in the oil and gas industry.

If gas hydrates are allowed to form inside a pipe containing natural gas and/or other petroleum fluids, they can eventually block the pipe. The hydrate blockage can lead to a shutdown in production and significant financial loss. The oil and gas industry therefore uses various means to prevent the formation of hydrate blockages in pipelines. These include heating the pipe, reducing the pressure, removing the water and adding thermodynamic inhibitors (antifreezes) such as methanol and ethylene glycols, which act as melting point depressants. Each of these methods is costly to implement and maintain. The most common method used today is the addition of antifreezes. However, these antifreezes have to be added at high concentrations, typically 10-60% by weight of the water present, in order to be effective. Recovery of the antifreeze is also often required and is a costly procedure.

Because of the high CAPEX and/or OPEX costs there is a need for chemicals that work at lower concentrations. This led to the development of low dosage hydrate inhibitors (LDHIs) which control the gas hydrate formation process using nucleation and crystal growth inhibitors. The advantage of using these chemicals to control gas hydrate formation is that they can be used at concentrations of 0.01 to 3%, i.e. much lower than concentrations typically used for antifreezes. LDHIs are divided essentially into two categories, kinetic hydrate inhibitors (KHIs) and anti-agglomerants (AAs).

Gas hydrate nucleation inhibitors are called kinetic hydrate inhibitors (KHIs). KHI polymers are often expensive, therefore a lower concentration of KHI polymer (perhaps 40-60% as much) is often used with the addition of a cheaper synergist to improve the performance and lower the overall cost.

Some kinetic hydrate inhibitor polymers cannot be used on some oil/gas fields because they have a cloud point (or lower critical solution temperature) in the produced aqueous fluid below the temperature where the polymer would be injected, e.g. at the wellhead. This would cause the polymer to deposit near the injection point, rendering it ineffective for the job for which it was designed. It could also cause a restriction in the conduit near the injection point. It would therefore be advantageous if alternative additives could be found. Most KHIs are limited in their use to subcoolings of 10-12° C.

Besides KHIs, there is another class of LDHIs called anti-agglomerants (AAs). Anti-agglomerants are surfactants and can be used at higher subcoolings than KHIs. AAs do not inhibit the formation of gas hydrates to the same level as KHIs, rather their primary activity is in preventing the agglomeration and deposition of hydrate crystals. A hydrocarbon phase provides a transport medium for the hydrates which are referred to as hydrate slurries so that the overall viscosity of the medium is kept low and can be transported along the pipeline. As such, the hydrate crystals formed in the water-droplets are prevented from agglomerating into a larger crystalline mass. Chemicals acting as anti-agglomerate hydrate inhibitors are typically quaternary ammonium or phosphonium salts, such as tributylhexadecylphosphonium bromide and tributylhexadecylammonium bromide.

Unfortunately, such compounds have undesirable levels of toxicity, are poorly biodegradable and are unable to function well in water with relatively low salt concentrations (such as some areas of the North Sea). Also, some AAs decrease the quality of the overboard discharged produced water, requiring extra treatment to reach the local residual oil-in-water regulations. There is clearly a need for AAs that can overcome some of these challenges.

Due to the above-mentioned problems relating to cost, performance and environmental impact, a need exists for alternative compounds for inhibiting and controlling the formation and agglomeration of gas hydrates in connection with hydrocarbon production, storage and transportation including production, drilling, completion, fracturing, stimulation and injection and reinjection operations.

It is therefore an object of the present invention to find novel and effective compounds which retard the formation of gas hydrates (kinetic inhibitors) or keep the gas hydrate crystals small and pumpable (anti-agglomerants).

It has been surprisingly found that certain compounds can inhibit the formation of hydrates and/or prevent agglomeration of hydrate crystals, while exhibiting improved biodegradability in comparison to existing additives. Thus, the present invention provides alternative compounds for inhibiting and controlling the formation of gas hydrates in connection with hydrocarbon production, storage and transportation including production, drilling, completion, fracturing, stimulation and injection and reinjection operations. The compounds can act as synergists for new or existing KHI polymers, as anti-agglomerants and as kinetic hydrate inhibitors themselves.

This, viewed from a first aspect, the present invention provides a method for inhibiting the formation or agglomeration of gas hydrates in a system, said method comprising adding to the system a compound of formula (I) or a zwitterionic form thereof:

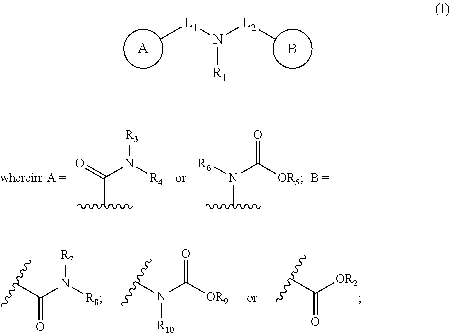

R₁ is a group comprising 4 to 28 carbon atoms and at least one C═C double bond;
L₁ and L₂ are independently selected from bonds and linker moieties containing 1 to 12 carbon atoms; and
R₂, R₃, R₄, R₅, R₆, R₇, R₈, R₉ and R₁₀ are independently H or a group comprising 1-20 carbon atoms.

Zwitterionic forms may be represented by the following formula (Z):

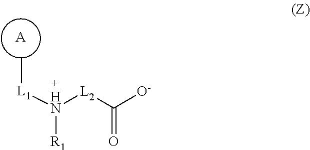

Thus, compounds of Formula (I) and zwitterionic forms are represented below:

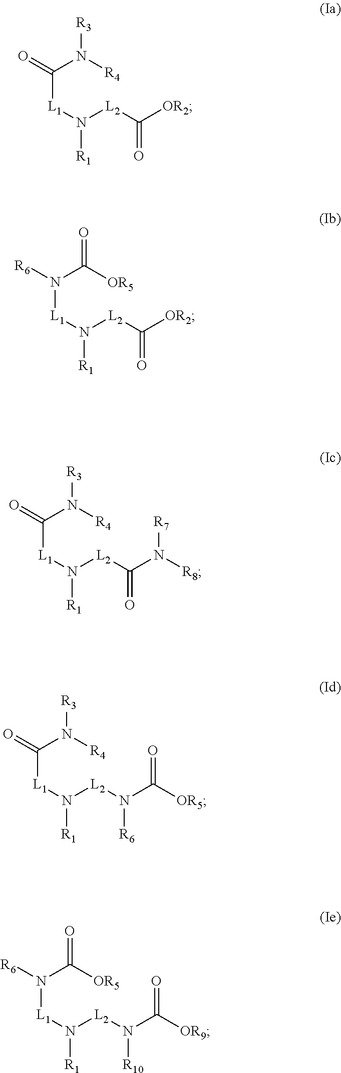

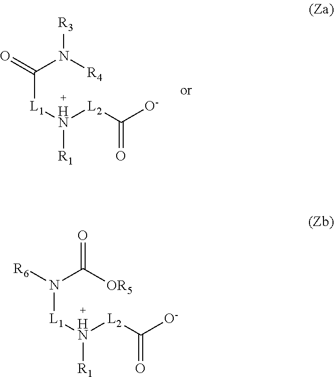

One or more of L₁, L₂, R₂, R₃, R₄, R₅, R₆, R₇, R₈, R₉ and/or R₁₀ may contain one or more heteroatoms. One or more of L₁, L₂, R₂, R₃, R₄, R₅, R₆, R₇, R₈, R₉ and/or R₁₀ may be or comprise an aromatic group, however, preferably they are aliphatic. In some embodiments, one or more of L₁, L₂, R₂, R₃, R₄, R₅, R₆, R₇, R₈, R₉ and/or R₁₀ are hydrocarbyl groups, i.e. they consist of carbon and hydrogen.

The present invention provides the first ever anti-agglomerate with reasonable performance that is considered, under Norwegian regulations, to be acceptable for use offshore due to its good biodegradation characteristics. Without wishing to be bound by theory, it is thought that the improved biodegradation is obtained due to the unsaturated nature of the tail group, i.e. R₁. An example is the oleyl tail which is found in many natural oils. In combination with the specific functional head group shown in Formulae (I) and (Z), the compounds work as an anti-agglomerant, while also being suitably biodegradable. The compounds and compositions described herein may therefore be described as biodegradable hydrate inhibitors or biodegradable anti-agglomerants.

Typically R₁ is a hydrophobic group. R₁ may be or comprise an aromatic group (Ar), however, preferably it is aliphatic. Preferably R₁ is an aliphatic group. Due to the presence of at least one C═C double bond (which may be cis or trans in configuration), R₁ is at least partially unsaturated. R₁ may comprise more than 1, e.g. 2 or more, preferably 1 to 6, especially 1 to 3, C═C double bonds. One or more carbon-to-carbon triple bonds may be present. R₁ may be branched and/or substituted, however, it is preferably an unsubstituted, unbranched (e.g. linear) group. Preferably R₁ comprises 6 to 24 carbon atoms, especially 12 to 18, e.g. 16 to 18 carbon atoms. Typically, R₁ is a fatty acid residue (i.e. the hydrocarbyl chain remaining when the acid group is removed from a fatty acid), for example the hydrocarbyl chain derived from one of the following fatty acids and their isomers:

| Fatty acid | “Residue” (R₁) |
|---|---|
| Myristoleic acid | CH₃(CH₂)₃CH═CH(CH₂)₇CH₂— |
| Palmitoleic acid | CH₃(CH₂)₅CH═CH(CH₂)₇CH₂— |
| Sapienic acid | CH₃(CH₂)₈CH═CH(CH₂)₄CH₂— |
| Oleic acid | CH₃(CH₂)₇CH═CH(CH₂)₇CH₂— (i.e. “oleyl”) |
| Elaidic acid | CH₃(CH₂)₇CH═CH(CH₂)₇CH₂— |
| Vaccenic acid | CH₃(CH₂)₅CH═CH(CH₂)₉CH₂— |
| Linoleic acid | CH₃(CH₂)₄CH═CHCH₂CH═CH(CH₂)₇CH₂— |
| Linoelaidic acid | CH₃(CH₂)₄CH═CHCH₂CH═CH(CH₂)₇CH₂— |
| α-Linoleic acid | CH₃CH₂CH═CHCH₂CH═CHCH₂CH═CH(CH₂)₇CH₂— |
| Arachidonic acid | CH₃(CH₂)₄CH═CHCH₂CH═CHCH₂CH═CHCH₂CH═CH(CH₂)₃CH₂— |
| Eicosapentaenoic | CH₃CH₂CH═CHCH₂CH═CHCH₂CH═CHCH₂CH═CHCH₂CH═CH(CH₂)₃CH₂— |
| acid | |
| Erucic acid | CH₃(CH₂)₇CH═CH(CH₂)₁₁CH₂— |
| Docosahexaenoic | CH₃CH₂CH═CHCH₂CH═CHCH₂CH═CHCH₂CH═CHCH₂CH═CHCH₂CH═CH(CH₂)₂CH₂— |
| acid | |

In a particularly preferred aspect, R₁ is CH₃(CH₂)₇CH═CH(CH₂)₇CH₂—, i.e. an “oleyl” group.

Each “L” is a bond or a linker (e.g. divalent) group containing 1 to 12 carbon atoms, preferably 1 to 6, especially 1 to 3 carbon atoms. L may be or comprise an aromatic group (Ar), but is preferably aliphatic. L₁ and L₂ may be the same or different. In a preferred embodiment, L₁ and L₂ are identical. These linker moieties may be branched, unsaturated and/or substituted. Preferably, the one or both of the linker groups is unsubstituted, or only substituted with alkyl groups (i.e. branched). Preferably one or both of the linker groups is saturated. Alkylene groups (linear or branched) are especially preferred.

In an embodiment, L₁ and L₂ are independently selected from —(CH₂)ₙ— or —(CH₂C(CH₃)H)—, where n=1-6, preferably, 1-3, e.g. 1 or 2. When L=—(CH₂C(CH₃)H)—, the CH₂ is preferably nearest the R₁—N—. Particularly preferably, L₁ and/or L₂ (preferably both) are —CH₂— or —CH₂—CH₂—.

R₂, R₃, R₄, R₅, R₆, R₇, R₈, R₉ and R₁₀ are independently H or a group comprising 1-20 carbon atoms and optionally one or more heteroatoms. One or more of R₂, R₃, R₄, R₅, R₆, R₇, R₈, R₉ and R₁₀ may be or comprise an aromatic group (Ar), but is preferably aliphatic. Preferably, R₂, R₃, R₄, R₅, R₆, R₇, R₈, R₉ and R₁₀ are independently selected from C₁₋₂₀ (especially C₁₋₁₂, e.g. C₁₋₈) groups (especially aliphatic groups) or hydrogen. The aliphatic groups are preferably (linear or branched) alkyl groups.

R₂, R₃, R₄, R₅, R₆, R₇, R₈, R₉ and R₁₀ are independently optionally branched, optionally unsaturated and/or optionally substituted.

In some embodiments, R₃ and R₇ are identical. In some embodiments, R₄ and R₈ are identical. In some embodiments, R₂ and R₅ are identical. In some embodiments, R₆ and R₁₀ are identical. In some embodiments, R₅ and R₉ are identical. In some embodiments, R₂, R₅ and R₉ are identical.

In a preferred embodiment, one or more of R₂, R₃, R₄, R₅, R₆, R₇, R₈, R₉ and R₁₀ is C₁₋₆ alkyl or hydrogen, preferably hydrogen.

Especially preferably R₃ (and/or R₇) is isopropyl and R₄ (and/or R₈) is hydrogen, or vice versa.

R₂, R₅, R₆, R₉ and/or R₁₀ are preferably hydrogen or methyl, especially hydrogen. R₂, R₅, and/or R₉ are preferably hydrogen or methyl, especially hydrogen.

As used herein, by the term “alkyl” is meant linear or branched alkyl group containing the recited number of carbon atoms. Preferably the alkyl groups are unsubstituted.

As used herein the term “C₁₋₆ alkyl” refers to any straight-chain or branched alkyl group having one, two, three, four, five or six carbon atoms, such as methyl, ethyl, n-propyl, isopropyl, n-butyl, iso-butyl, sec-butyl, t-butyl, n-pentyl, tert-pentyl, neopentyl, isopentyl, sec-pentyl, 3-pentyl, 1-hexyl, 2-hexyl or 3-hexyl groups. C₁₋₄, e.g. C₂₋₃ alkyl groups (e.g. isopropyl), are especially preferred.

Any aromatic moiety (Ar) is independently selected from substituted and substituted single ring (e.g. phenyl or phenylene) and substituted and unsubstituted polynuclear/polycyclic aromatic moieties. The term “polynuclear” is considered to encompass fused aromatic rings such as naphthalene and non-fused rings such as biphenyl, etc. Particularly preferably, Ar is phenyl or phenylene.

Where substituents are mentioned (with respect to any moiety, aliphatic or aromatic), these are typically selected from, for example, hydroxyl, alkoxy, alkyl (e.g. C₁₋₆ alkyl) groups and the like. A “branched” group as herein described may be considered to be equivalent to one substituted with an alkyl group.

In some embodiments, the compound of the invention (i.e. the compound for use in the methods and uses described herein, in addition to the compound per se and compositions comprising it) is selected from those according to Formulae (Ia), (Ib), (Ic), (Za) and (Zb).

In some embodiments, the compound of the invention is selected from those according to Formulae (Ia), (Ib), (Za) and (Zb).

In some embodiments, the compound of the invention is selected from those according to Formulae (Ia) and (Ib).

In a preferred embodiment, the compound of Formula (I) is one according to Formula (Ia) (or a zwitterionic form thereof):

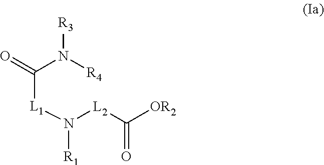

Especially preferred compounds are those according to the following formula, where R₁, R₃ and R₄ are as described herein (or a zwitterionic form thereof):

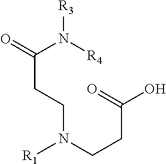

A particularly preferred compound according to the present invention is the following, denoted herein as “IPOI” (where “oleyl=CH₃(CH₂)₇CH═CH(CH₂)₇CH₂—):

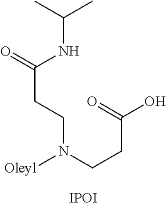

Viewed from a further aspect, the invention provides the use of a compound as herein defined for inhibiting the formation or agglomeration of hydrates in a system, preferably a system for hydrocarbon drilling, production, storage and/or transportation, including production, drilling, completion, fracturing, stimulation and injection and re-injection operations.

Compositions comprising the compounds described herein form a further embodiment of the invention. Said compositions are also applicable to the methods and uses described herein.

More than one compound as described herein may be added to the system in the method and uses of the invention. For example, mixtures of two or more of the compounds as herein described may be used in the methods and uses herein described, or may be present in the compositions of the invention.

The compounds as herein described have been found to be particularly effective in fresh water or low salinity water. This is advantageous because many conventional anti-agglomerants are ineffective in conditions of low salinity.

If necessary, an additive may be used, e.g. to improve the performance of the compounds of the invention, e.g. in saline water. Suitable compounds for this purpose are acids, e.g. di-acids, polyacids, organic sulfonic acids, organic phosphoric acids e.g. biodegradable polyacids. Examples include citric acid or tartaric acid, which are also biodegradable natural chemicals. The ratio of the weight % of the acid to that of the compound of the invention (e.g. in a method, use, or composition described herein) is typically in the range of 10:1 to 1:10, e.g. 5:1 to 1:5, approximately 1:1.

It has further been surprisingly found that the compounds herein described (i.e. compounds according to Formula (I) or zwitterionic forms thereof) can be used in conjunction with thermodynamic hydrate inhibitors (THIs) such as MEG. These combinations have been found to give a surprising synergistic improvement in AA performance, particularly under more extreme conditions (higher subcooling, shut-in/start-up etc.). The compounds according to Formula (I), or zwitterionic forms thereof, thus exhibit synergy and can be termed “synergistic agents”.

The effect of combining two types of active agent would be expected to be additive, e.g. if the AA alone in a particular system is capable of performing to a maximum of 12° C., the addition of sufficient MEG to lower the system equilibrium temperature by 2° C. would be expected to, at most, allow the AA to be used at 12+2=14° C. subcooling. However, it has been found that the effect is synergistic for THIs with compounds of the present invention. This decreases the active concentration at high subcoolings.

Thus, the compounds of the present invention (i.e. compounds according to Formula (I) or zwitterionic forms thereof) may be used in combination with one or more of an acid (e.g. as described above) and a thermodynamic hydrate inhibitor. These combinations apply to the compositions, methods and uses herein described. Suitable acids include polyacids such as citric acid or tartaric acid. Suitable thermodynamic hydrate inhibitors include glycols, e.g. monoethylene glycol (MEG), diethylene glycol (DEG) and triethylene glycol (TEG).

Synergistic combinations of compounds according to the present invention (i.e. compounds according to Formula (I) or zwitterionic forms thereof) and thermodynamic hydrate inhibitors form a further aspect of the present invention. Compositions comprising the synergistic combinations and uses of said compositions/combinations in the methods and uses herein described also form part of the present invention. Synergistic combinations are considered to be those which are capable of performing to a certain standard at a higher temperature than the additive effect of the components. Example 3 shows this effect for IPOI and MEG, a particularly preferred combination according to the present invention. Methods for enhancing (e.g. synergistically improving) the performance of a THI comprising using said THI in conjunction with, or combining said THI with, a compound as described herein are thus also provided.

When a THI is present in combination (e.g. in a method, use, or composition described herein) with a compound according the present invention (i.e. a compound according to Formula (I) or a zwitterionic form thereof), the ratio of the weight % of the thermodynamic hydrate inhibitor to that of the compound of the invention is typically in the range of 100:1 to 1:10, e.g. 50:1 to 1:5, approximately 10:1.

The compounds as described herein (i.e. a compound according to Formula (I) or a zwitterionic form thereof) can be used as hydrate inhibitors (e.g. anti-agglomerants) themselves or as synergists (performance enhancing chemicals) for new and existing hydrate inhibitors, i.e. KHI polymers. The methods (or uses or compositions) of the invention may therefore further comprise adding a hydrate inhibitor (e.g. a kinetic hydrate inhibitor and/or a thermodynamic hydrate inhibitor), e.g. to the system. Use of the compounds herein described as hydrate inhibitor synergists forms a further embodiment of the invention.

In some embodiments, the method of the present invention is a method for inhibiting agglomeration of gas hydrates. The compounds according to Formula (I) or zwitterionic forms thereof may be considered to be anti-agglomerants (AAs) and the compositions may be consider to be anti-agglomerant compositions.

Thus viewed from a further aspect, the present invention provides the use of a compound or composition as herein described as a hydrate anti-agglomerant.

The compositions, methods and uses of the invention are applicable to any system or situation in which gas hydrate formation and/or agglomeration is desired to be controlled. In particular, they are applicable to systems for hydrocarbon drilling, production, storage and/or transportation, including production, drilling, completion, fracturing, stimulation and injection and re-injection operations. Typically, the “system” referred to herein is a fluid and/or a conduit.

Addition of the compounds to the system may be achieved through any known means and in amounts typical in the art. However, due to the surprising efficacy of the compounds of the invention, lower amounts may be required than of conventional hydrate inhibitor or anti-agglomerant compounds. Typical use concentrations, calculated as 100% of active substance (e.g. compound according to Formula (I) or a zwitterionic form thereof), are 0.005 to 8%, preferably 0.0075 to 5%, more especially 0.01 to 3% especially concentrations of from 0.02 to 1 wt % (100-10,000 ppm) by weight based on the water present in the system.

The present invention is useful for inhibiting hydrate formation or inhibiting agglomeration of hydrates for many hydrocarbons and hydrocarbon mixtures, e.g. those which include methane, ethane, propane, n-butane, isobutane, isopentane and mixtures thereof. Other examples include various natural gas mixtures that are present in many gas and/or oil formations and natural gas liquids (NGL). The hydrates of all of these low-boiling hydrocarbons are also referred to as gas hydrates. The hydrocarbons may also comprise other compounds including, but not limited to, CO₂, hydrogen sulphide, and other compounds commonly found in gas/oil formations or processing plants, either naturally occurring or used in recovering/processing hydrocarbons from the formation or both, and mixtures thereof.

The methods and uses of the present invention involve contacting a hydrocarbon and water mixture with a compound or composition as described herein. When an effective amount of the compound/composition is used, hydrate blockage is inhibited. The contacting may be achieved by means of standard equipment such as injection pumps or the like, resulting in rapid and uniform distribution of the inhibitor in the aqueous phase which has a tendency to form hydrates.

The contacting can be made in-line or offline or both. When the compounds of the invention are added in a composition, the various components of the composition may be mixed prior to or during contact, or both. If needed or desired, the composition or some of its components may be optionally removed or separated mechanically, chemically, or by other methods known to one skilled in the art, or by a combination of these methods after the hydrate formation or agglomeration conditions are no longer present.

The pressure at which the compounds/compositions are contacted with the hydrocarbon/water mixture is usually at, or greater than, atmospheric pressure. (i.e. about 101 kPa), preferably greater than about 1 MPa, and more preferably greater than about 5 MPa. The pressure in certain formation or processing plants or units could be much higher, for example greater than about 20 MPa. There is no specific high-pressure limit. The present invention can be used at any pressure that allows formation of hydrocarbon gas hydrates.

Since the inhibitor primarily retards or prevents the formation of gas hydrates, the addition of the inhibitor should ideally take place before gas hydrates are formed, i.e. at above the equilibrium temperature of hydrate formation. The temperature for contacting is usually below, the same as, or not much higher than, the ambient or room temperature. Lower temperatures tend to favour hydrate formation, thus requiring the treatment with the compositions/compounds of the present invention. For anti-agglomerant applications, the compounds or compositions may be added before, during, or after hydrate formation, preferably before.

In the methods and uses of the present invention, the compounds and compositions herein described may be added to the system at any stage or location suitable to inhibit formation or agglomeration of hydrates. The conduits into which the compounds/composition of the invention are added are typically hydrocarbon conduits extending for at least part of the length from the site within a hydrocarbon well at which hydrocarbon enters the borehole to the facility remote from the well at which hydrocarbon compositions are processed. Typically, the compounds/compositions are added to a process stream containing hydrocarbons and water by injection via a single port or multiple ports. In one aspect, the compound may be injected into the reservoir matrix surrounding a hydrocarbon production well. In a further aspect, the compound may be injected into a hydrocarbon production well. Preferably, the compound is injected at the well head.

The compounds of the invention may be used alone or together with a further component, such as a hydrate inhibitor, a liquid solvent, a solid carrier and/or an excipient.

A further embodiment of the invention is the provision of hydrate inhibitor or anti-agglomerant compositions. Thus from a further aspect, the present invention provides a hydrate inhibitor or anti-agglomerant composition comprising a compound as herein described (i.e. a compound according to Formula (I) or a zwitterionic form thereof). Optionally, the composition further comprises an acid as described above, a thermodynamic hydrate inhibitor as described above, a kinetic hydrate inhibitor, a solvent (e.g. a liquid solvent), a carrier (e.g. a solid carrier) and/or an excipient. In a particularly preferred aspect, the composition of the invention is a hydrate inhibitor composition comprising a kinetic hydrate inhibitor together with a compound as herein described (i.e. a compound according to Formula (I) or a zwitterionic form thereof). The compounds and compositions may be used in the methods and uses described herein.

Further preferred additives for use together with the compounds of the invention, in the methods, uses and compositions of the invention, include polymers, amphiphiles and surfactants. These may be non-ionic or anionic. Examples are alkylpolyglycosides, hydroxylethylcellulose, carboxymethylcellulose and other ionic or nonionic surfactant molecules. Especially preferred are anionic surfactants. Other suitable additives are corrosion inhibitors and scale inhibitors.

Suitable solvents, carriers and excipients are known in the art and include oxygenated solvents such as water, alcohols, ether solvents and mixtures thereof. Solvents, carriers or excipients are typically present in the (inhibitor) compositions in the range from 0 wt % to 95 wt %, e.g. 20 wt % to 95 wt %, preferably 50 wt % to 95 wt % of the total composition.

When KHIs are used, preferably the kinetic hydrate inhibitor polymer is a polymer, copolymer or graft polymer prepared from or one or more N-vinyl lactams, N-alkylacrylamides, N,N-dialkylacrylamide, N-alkylacrylamides, N,N-dialkylacrylamide, N-vinyl-N-alkyl alkanamides, or a hyperbranched poly(esteramide), or a peptide or protein including polyaspartamides or a polymer or copolymer containing pyroglutamate groups. Especially preferably the KHI is a polyvinyllactam. The ratio of kinetic hydrate inhibitor to compound of the invention is preferably from 95:5 to 10:90 by weight.

Certain of the compounds herein described are novel and thus form a further aspect of the present invention. Thus, viewed from a further aspect, the present invention provides the compounds as herein described (i.e. compounds according to Formula (I) or zwitterionic forms thereof) and compositions comprising said compounds.

Tautomers, enantiomers, diastereomers, analogues, isomers, ions, salts and mixtures of the compounds herein described (i.e. compounds according to Formula (I) or zwitterionic forms thereof) are also applicable to the invention as herein described.

Preparation of the compounds of the invention is possible using techniques known in the art. For example, to prepare IPOI, oleylamine or an equivalent primary amine mixture made from an oil source high in oleyl groups (e.g. sunflower oil) can be used. Thus a method for preparing a compound as herein described comprises reaction of R₁—NH₂ (where R₁ is as described herein) with acrylic acid and reacting the resulting product with N-isopropylacrylamide. The reactants should preferably be used in equimolar amounts.

A suitable synthesis method for IPOI is set out below:

1. Add (e.g. via stirring) acrylic acid (or 80% acrylic in water) and phenothiazine (optional) into a suitable solvent (e.g. methanol or other alcohol or glycol or glycol ether).

2. Cool the mixture (e.g. by ice bath) and maintain the temperature below 30° C.

3. Add a solution (e.g. a 50 wt % solution) of a suitable amine (in this case oleyl amine) e.g. drop-wise for 1 hour.

4. Heat to around 80° C. and maintain at this temperature, e.g. for 4 hours.

5. Remove the solvent (e.g. by rotary evaporation) to obtain N-oleyl-beta-alanine.

6. Dissolve N-oleyl-beta-alanine and N-isopropylacrylamide in a suitable solvent (e.g. isopropanol or other alcohol, glycol or glycol ether).

7. Heat to around 90° C. and keep at that temperature for around 8 hours.

8. Remove the solvent to produce IPOI.

The compounds described herein may also be used to protect against corrosion, i.e. in some cases it may be unnecessary to use another molecule as a specific corrosion inhibitor if the compounds of this invention can do the job. Alternatively, less corrosion inhibitor may be necessary due to the partial protection provided by the compounds of the invention. The compounds described herein may also have biocidal or scale inhibition properties.

Thus, from a further aspect, the present invention provides the use of a compound as herein defined as a corrosion inhibitor, a biocide or a scale inhibitor.

All references herein to “comprising” should be understood to encompass “including” and “containing” as well as “consisting of” and “consisting essentially of”.

The invention will now be further described with reference to the following non-limiting examples:

### EXAMPLE 1—PREPARATION OF IPOI

Step 1: 1 mole of acrylic acid (or 80% acrylic in water) and 0.2 g of phenothiazine (optional) are added into 100 ml methanol (or other alcohol or glycol or glycol ether) and cooled by ice bath and the temperature kept lower than 30° C. To that solution, 1 mole of oleylamine (e.g. as 50 wt % solution) is added drop-wise for 1 hour. After addition it is heated up to 80° C. and kept at 80° C. for 4 hours. Removing methanol by rotary evaporator, N-oleyl-beta-alanine is obtained.

Step 2: Equimolar amounts of N-dodecyl-beta-alanine and N-isopropylacrylamide are dissolved in 100 ml of isopropanol (or other alcohol, glycol or glycol ether) and then the solution is heated up to 90° C. and kept at that temperature for 8 hours. Removing isopropanol, IPOI is obtained.

### EXAMPLE 2—BIODEGRADATION

The following table shows Ecotox (ecotoxicology) data for IPOI and its coco derivative, FX-IPC. Ecotox is made up of three basic tests:
(i) Biodegradation—for offshore use, the OECD seawater test in Europe is called OECD306. It measures how much percentage of a chemical has degraded in 28 days, “BOD28”. If needed the test can be prolonged to 60 days to give “BOD60” values.
(ii) Bioaccumulation—this is OECD117 and measures the logPow value which is the log of the ratio of the distribution of a chemical between octanol and water. High values, especially over 3, are undesirable as this indicates strong uptake of the chemical into a species where it can cause most damage.
(iii) Acute toxicity—this is measured on several species, such as skeletonema (usually the most sensitive species and used as a first screening of the toxicity of a chemical), acartia, daphnia etc. Chronic, long-term effects are not yet part of any offshore regulations.

These three Ecotox tests are used to categorise the hazards of a chemical to be discharged into a body of water. In the North Sea, Norway and Denmark use the Ecotox regulations somewhat strictly. Many offshore areas have no regulations, and thus, while the North Sea regulations are comparatively environmentally-friendly, they are not compulsory. In the Gulf of Mexico, where AAs are used more than any other area, the environmental regulations are based solely on acute toxicity and the dilution factor away from the platform. This has allowed the use of the toxic cationic surfactant AAs which dominate the AA market today, none of which are allowed in the North Sea due to high toxicity and low biodegradability. Norway focuses greatly on having chemicals with high biodegradation because then they know the chemical will have little impact after 28 days, irrespective of toxicity, if it degrades fast. BOD28>60% is the target, which is very hard to reach for new chemicals. In the UK, 20-60% is often deemed acceptable as long as toxicity is not too high.

| Ecotox data for FX-IPC | |
|---|---|
| (comparative) | Ecotox data for IPOI |
| BOD28 = 57-61% | BOD28 = 62%* = |
| (UiS) = 49%* | 61-65% (UiS) |
| BOD60 = 70%* | BOD59 = 79%* |
| Mw < 700 | Mw < 700 |
| Log Pow 1.3* | Log Pow 2.3* |
| EC50 0.1-1 mg/litre | EC50 0.1-1 mg/litre* |

(“UiS” denotes test carried out by the University of Stavanger; *denotes that the test was carried out by a certified authority)

FX-IPC (a derivative of IPOI where R₁ coco (saturated C₁₂₋₁₄ mainly) showed good anti-agglomerant performance. However, the biodegradation of FX-IPC was less than 60% in 28 days (by OECD306) and the toxicity high, meaning that FX-IPC would be categorized Yellow 2 or red in Norway and would not be allowed for use offshore. Also, FX-IPC did not have good performance in fresh water, only saline solutions.

Replacing the tail (R₁) with an oleyl group (to make IPOI) gave an AA with over 60% biodegradation in 28 days and a log Pow of 2.3, giving an environmental category of Yellow 1 in Norway, which is acceptable. This may be a function of the unsaturated C═C double bond in the oleyl group. IPOI also performs well as an AA in freshwater whereas FX-IPC did not.

### EXAMPLE 3—ANTI-AGGLOMERANT TESTS USING IPOI

All anti-agglomerant tests were carried out in sapphire rocking cells at 80 or 120 bar. Either deionized water (DW) or a brine of 4.88 wt. % NaCl and 0.35 wt. % CaCl₂ (i.e. TDS=5.23 wt. %) was used.

Two types of tests were carried out:
(i) C.C.=constant cooling test, cooling at the same rate of 5° C./h before reaching 2° C. where the cells were rocked continuously for at least 4 hours.
(ii) Extreme shut-in test. The cell was cooled at 5° C./h to a set temperature, usually 4 or 2° C. This is ca. 12 and 14° C. subcooling at 80 bar for deionized water. The cells were rocked until a little hydrate is formed, usually with about 1 bar pressure drop. The cells were then shut in for 4 hours at the set temperature with no rocking and then restarted. This extreme test with some preformed hydrates is harder to pass than a standard shut-in/start-up test in which the cells are shut in without prior formation of gas hydrates.

In Table 1, ranking of performance varies from A to E. A is best, showing a fine dispersion of very small hydrate particles and no deposits. B=almost the same with slightly larger particles. C═some particles have caused deposits but which are dispersed later. D=solid deposits build up. E=rapid plug of hydrates formed. When varying results were observed, the worst-case result is shown.

TABLE 1
Tests in sapphire rocking cells with sII-forming natural
gas mixture, a condensate and aqueous fluid giving a water
cut of 15%. C.C. = constant cooling at 5° C./h.
Extreme = shut-in at 2° C. (ca. 14° C. subcooling
at 80 bar for DI water) with some preformed hydrates, then
start-up 4 h later. 1 wt. % IPOI based on the water phase
was used in all tests. [Acid] = tartaric acid.

| | [Acid] | [MEG] | Pressure | TDS | | Performance |
|---|---|---|---|---|---|---|
| Entry | Wt. % | Wt. % | bar | Wt. % | Test | Ranking |
| 1 | — | — | 80 | — | C.C. | A |
| 2 | — | — | 80 | 5.23 | C.C. | D |
| 3 | 1 | — | 80 | 5.23 | C.C. | A |
| 4 | — | — | 80 | — | Extreme | E |
| 5 | — | — | 80 | 5.23 | Extreme | E |
| 6 | — | 10 | 80 | — | Extreme | B* (A at 4° C.) |
| 7 | 1 | — | 80 | 5.23 | Extreme | B* (A at 4° C.) |
| 8 | 1 | 10 | 80 | 5.23 | Extreme | A |

TABLE 2
Tests in sapphire rocking cells with sII-forming natural gas mixture,
a plugging North Sea oil and aqueous fluid giving a water cut of 15%.
C.C. = constant cooling at 5° C./h. Extreme = shut-in at 2° C.
(ca. 14° C. subcooling at 80 bar and ca. 16° C. subcooling at 120 bar
for deionized water) with some preformed hydrates, then start-up 4 hours
later. 1 wt. % IPOI based on the water phase was used in all tests.
[Acid] = tartaric acid.

| | | | | TDS | | |
|---|---|---|---|---|---|---|
| | [Acid] | [MEG] | Pressure | Wt. | | Performance |
| Entry | Wt. % | Wt. % | bar | % | Test | Ranking |
| 1 | — | — | 80 | — | C.C. | A |
| 2 | — | — | 80 | 5.23 | C.C. | A |
| 3 | — | 10 | 80 | 5.23 | C.C. | A |
| 4 | 1 | — | 80 | 5.23 | C.C. | A |
| 5 | — | 10 | 80 | — | C.C. | A |
| 6 | 1 | 10 | 80 | 5.23 | C.C. | A |
| 7 | — | — | 80 | — | Extreme | C |
| 8 | — | 10 | 80 | — | Extreme | A |
| 9 | — | — | 80 | 5.23 | Extreme | D |
| 10 | 1 | — | 80 | 5.23 | Extreme | C* (B at 4° C.) |
| 11 | 1 | 10 | 80 | 5.23 | Extreme | A |
| 12 | — | — | 120 | — | C.C. | C |
| 13 | — | 10 | 120 | — | C.C. | A |
| 14 | — | — | 120 | 5.23 | C.C. | E |
| 15 | 1 | — | 120 | 5.23 | C.C. | A |
| 16 | | 10 | 120 | 5.23 | C.C. | A |
| 17 | 1 | 10 | 120 | 5.23 | C.C. | A |
| 18 | — | — | 120 | — | Extreme | E |
| 19 | — | 10 | 120 | — | Extreme | B# (A at 4° C.) |
| 20 | — | — | 120 | 5.23 | Extreme | D |
| 21 | 1 | | 120 | 5.23 | Extreme | D |
| 22 | | 10 | 120 | 5.23 | Extreme | A (4° C.), D (2° C.) |
| 23 | 1 | 10 | 120 | 5.23 | Extreme | A (4° C.), D (2° C.) |

1. A method for inhibiting the formation or agglomeration of gas hydrates in a system, said method comprising adding to the system a compound of formula (I) or a zwitterionic form thereof: 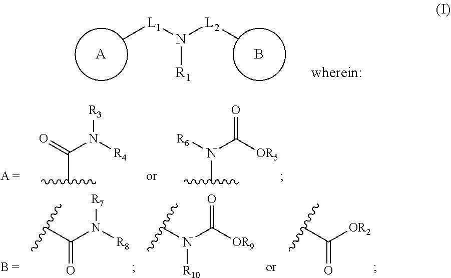
R₁ is a group comprising 4 to 28 carbon atoms and at least one C═C double bond;
L₁ and L₂ are independently selected from bonds and linker moieties containing 1 to 12 carbon atoms; and
R₂, R₃, R₄, R₅, R₆, R₇, R₈, R₉ and R₁₀ are independently H or a group comprising 1-20 carbon atoms.

2. The method as claimed in claim 1 wherein said compound is: 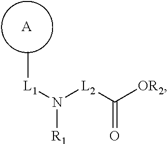
or a zwitterionic form thereof;

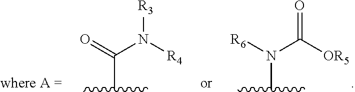

3. The method as claimed in claim 1, wherein R₁ comprises 6 to 24 carbon atoms.

4. The method as claimed in claim 1, wherein L₁ and L₂ are independently selected from —(CH₂)ₙ— or —(CH₂C(CH₃)H)—, where n=1-6.

5. The method as claimed in claim 1, wherein one or more of R₂, R₃, R₄, R₅, R₆, R₇, R₈, R₉, and R₁₀ is C₁₋₆ alkyl or hydrogen.

6. The method as claimed in claim 1, wherein R₃ and/or R₇ is isopropyl, and R₄ and/or R₈ is hydrogen and/or R₂, R₅, R₆, R₉ and/or R₁₀ is hydrogen.

7. The method as claimed in claim 1, wherein:

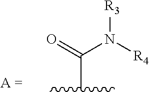

8. The method as claimed in claim 1, wherein said compound has the following formula:

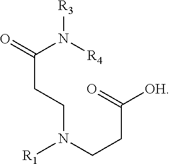

9. The method as claimed in claim 1, wherein said compound is:

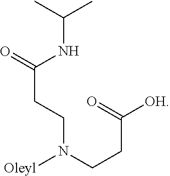

10. The method as claimed in claim 1, wherein said system is one for hydrocarbon drilling, production, storage and/or transportation, including production, drilling, completion, fracturing, stimulation and injection and re-injection operations.

11. The method as claimed in claim 1, wherein said method is for inhibiting the agglomeration of gas hydrates.

12. The method as claimed in claim 1, further comprising adding an acid and/or a thermodynamic hydrate inhibitor to said system.

13. A compound having formula (I). 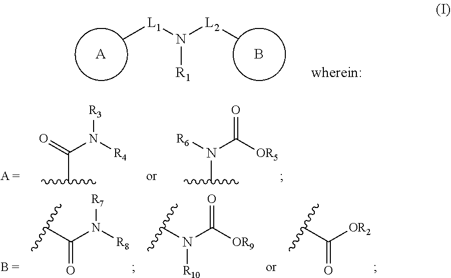
R₁ is a group comprising 4 to 28 carbon atoms and at least one C═C double bond;
L₁ and L₂ are independently selected from bonds and linker moieties containing 1 to 12 carbon atoms; and
R₂, R₃, R₄, R₅, R₆, R₇, R₈, R₉ and R₁₀ are independently H or a group comprising 1-20 carbon atoms, or a zwitterionic form thereof.

14. A composition comprising a compound as described in claim 13, said composition optionally further comprising an acid and/or a thermodynamic hydrate inhibitor.

15. (canceled)

16. The method as claimed in claim 1, wherein R₁ is an oleyl group.

17. The method as claimed in claim 4, wherein n=1-3.

18. The compound of claim 13, wherein said compound is: 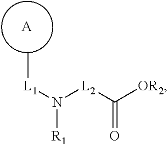
or a zwitterionic form thereof;

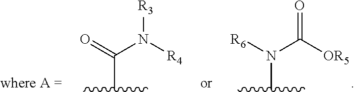

19. The compound of claim 13, wherein R₁ comprises 6 to 24 carbon atoms.

20. The compound of claim 13, wherein L₁ and L₂ are independently selected from —(CH₂)ₙ— or —(CH₂C(CH₃)H)—, where n=1-6.

21. The compound of claim 13, wherein one or more of R₂, R₃, R₄, R₅, R₆, R₇, R₈, R₉, and R₁₀ is C₁₋₆ alkyl or hydrogen.

22. The compound of claim 13, wherein R₃ and/or R₇ is isopropyl, R₄ and/or R₈ is hydrogen, and/or R₂, R₅, R₆, R₉ and/or R₁₀ is hydrogen.

23. The compound of claim 13, wherein:

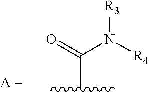

24. The compound of claim 13, wherein said compound has the following formula:

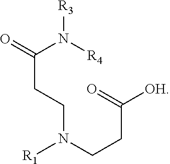

25. The compound of claim 13, wherein said compound is: [chemical structure]
